(19) United States
(12) Patent Application Publication
(10) Pub. No.: US 2011/0233146 A1
(43) Pub. Date: Sep. 29, 2011

(54) SYNERGISTIC WASTEWATER ODOR CONTROL COMPOSITION, SYSTEMS, AND RELATED METHODS THEREFOR

(75) Inventors: James Vaughan Harshman, Bradenton, FL (US); David Leonard Morano, Sarasota, FL (US)

(73) Assignee: Siemens Water Technologies Corp., Warrendale, PA (US)

(21) Appl. No.: 12/890,050

(22) Filed: Sep. 24, 2010

Related U.S. Application Data
(60) Provisional application No. 61/245,850, filed on Sep. 25, 2009.

(51) Int. Cl. C02F 1/72; A61L 101/32

(52) U.S. Cl. 210/758; 424/76.5

(57) ABSTRACT

Some aspects of the invention can involve compositions, systems, and related techniques that control or reduce objectionable odor characteristics of a body or a stream of wastewater. The compositions, systems, and related techniques can comprise one or more compounds that adjust metabolic activity of at least a portion of microorganisms in wastewater to inhibit or disfavor the formation of at least one objectionable odorous compound or species and one or more compounds that modify, shift, or promote one or more states or characteristics of one or more objectionable odorous species in wastewater. The metabolic modifying compound can be an anthraquinone and the state modifying compound can be an alkaline or pH-elevating compound.

### CROSS-REFERENCE TO RELATED APPLICATION

This application is a non-provisional application of and claims the benefit under 35 U.S.C. §119 of U.S. Patent Application No. 61/245,850, titled SYNERGISTIC EFFECT OF ANTHRAQUINONE AND ALKALINITY ENHANCING MATERIALS, filed on Sep. 25, 2009, which is incorporated herein by reference in its entirety for all purposes.

### BACKGROUND OF INVENTION

1. Field of Invention

This invention relates to compositions, systems and methods for controlling odor in wastewater, and, in particular, to systems and methods of odor control in sewerage systems by utilizing at least one alkaline compound and at least one metabolic modifier.

2. Discussion of Related Art

Sublette, in U.S. Pat. No. 5,480,550, discloses a biotreatment process for caustics containing inorganic sulfides.

Tatnall, in U.S. Pat. No. 5,500,368, discloses finely divided anthraquinone formulations that inhibit sulfide production by sulfate-reducing bacteria.

Miller et al., in U.S. Pat. No. 5,833,864, disclose a method for the reduction and control of the release of gas and odors from sewage and waste water.

Hunniford et al., in U.S. Pat. No. RE37,181 E, disclose a process for removal of dissolved hydrogen sulfide and reduction of sewage BOD in sewer or other waste systems.

### SUMMARY OF THE INVENTION

One or more aspects of the invention can relate to a method of controlling objectionable odor in a sewerage system. The method can comprise, consist of, or consist essentially of adding at least one alkaline compound to wastewater in the sewerage system, and at least one anthraquinone to the wastewater. A composition can be added as the at least one alkaline compound or as the at least one anthraquinone or with both. In one or more embodiments that can pertain to one or more aspects of the invention, the alkaline compound can be at least one hydroxide selected from the group consisting of alkali hydroxides, alkaline earth hydroxides, alkali earth oxides, and ammonium hydroxides. In one or more other embodiments that can pertain to one or more aspects of the invention, the anthraquinone can be 9,10-anthraquinone and, if appropriate, the alkaline compound can be at least one of sodium hydroxide, potassium hydroxide, calcium hydroxide, and magnesium hydroxide. In some embodiments related to some aspects of the invention, the anthraquinone can be at least one of 9,10-anthraquinone, a haloanthraquinone, an aminoanthraquinone, a hydroxyanthraquinone, and a nitroanthraquinone. One or more further embodiments related to some aspects of the invention can involve adding the at least one alkaline compound to the wastewater in an amount sufficient to raise the pH of at least a portion of the wastewater to be in a range that is at least about 8 units. One or more still further embodiments related to some aspects of the invention can involve adding the at least one alkaline compound to the wastewater in an amount sufficient to raise the pH of the at least a portion of the wastewater to be in a range of from about 8.2 to about 8.6. One or more further embodiments related to some aspects of the invention can involve adjusting a ratio of an amount of alkaline compound to an amount of the anthraquinone.

One or more aspects of the invention can relate to a wastewater stream comprising an odor controlling composition consisting essentially of an alkaline compound and an anthraquinone. In some embodiments of the wastewater stream, the alkaline compound can be at least one hydroxide selected from the group consisting of alkali hydroxides, alkaline earth hydroxides, alkali earth oxides, and ammonium hydroxides. In some embodiments of the wastewater stream of the invention, the anthraquinone can be at least one of 1,2-anthraquinone, 1,4-anthraquinone, and 2,6-anthraquinone, and 9,10-anthraquinone, 1-nitroanthraquinone, 1-chloroanthraquinone, 1-aminoanthraquinone, 1-hydroxyanthraquinone, 2-hydroxyanthraquinone, 2-aminoanthraquinone, 2-chloroanthraquinone, 1,5-dihydroxyanthraquinone, 2,6-dihydroxyanthraquinone, 1,8-dihydroxyanthraquinone, and 1,4-diaminoanthraquinone.

One or more aspects of the invention method facilitate odor control in a sewerage system. The method can comprise determining the presence of at least one odorous compound or species in the sewerage system, and providing an odor control composition consisting essentially of at least one alkaline compound and at least one anthraquinone. The method, in accordance with some embodiments for one or more aspects of the invention, can further comprise providing instructions to adjust the relative ratio of an amount of the at least one alkaline compound to an amount of the at least one anthraquinone.

### BRIEF DESCRIPTION OF THE DRAWINGS

The accompanying drawings are not drawn to scale. In the drawings, each identical or nearly identical component that is illustrated in the various figures is represented by a like numeral. For purposes of clarity, not every component may be labeled in every drawing.

In the drawings:

FIG. 1 is a flowchart showing of a control scheme which can be implemented in a control system in accordance with one or more aspects of the invention;

FIG. 2 is a depiction of a sewerage system with indicated nominal flow rates and associated treatment schemes prior to utilization of the compounds, compositions, systems, and methods in accordance with one or more aspects of the invention;

FIG. 3 is a depiction of the sewerage system with indicated nominal flow rates and associated treatment schemes with the compounds, compositions, systems, and methods in accordance with one or more aspects of the invention, as discussed in the Examples;

FIG. 4 is a graph showing the measured levels of hydrogen sulfide at various locations of the sewerage system schematically illustrated in FIG. 3 without utilizing the compounds, compositions, systems, and methods of the invention;

FIG. 5 is a graph showing the measured levels of hydrogen sulfide at various locations of the sewerage system schematically illustrated in FIG. 3 with and without utilizing the compounds, compositions, systems, and methods in accordance with one or more aspects of the invention; and

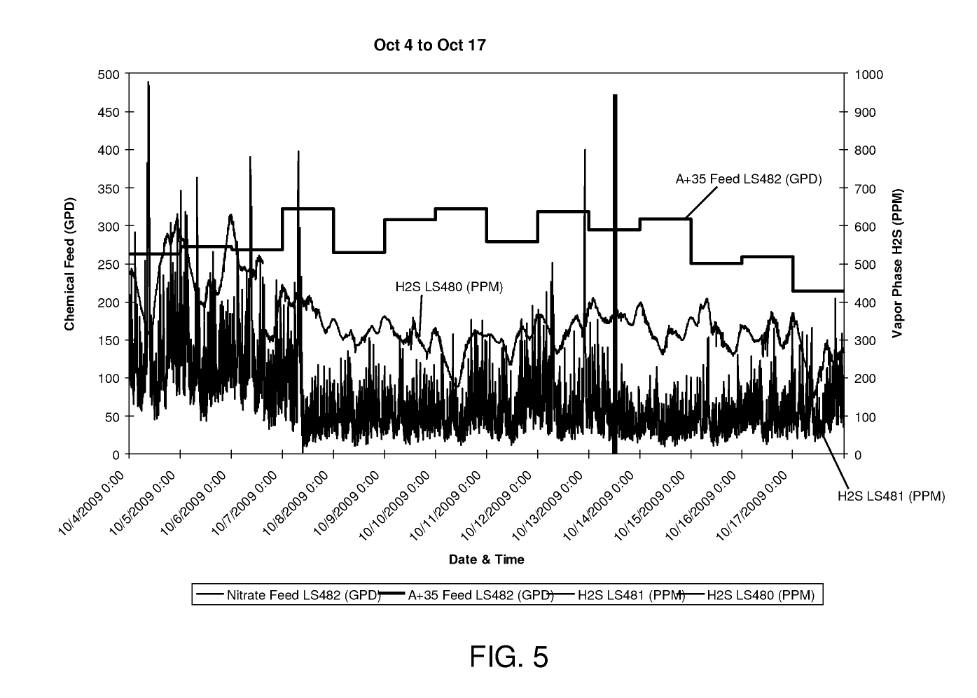

FIG. 6 is a graph showing the effect on hydrogen sulfide levels at various locations of the sewerage system depicted in FIG. 3 by utilizing calcium hydroxide slurry (A+) (nominally 25% solids) to control the on pH of the wastewater;

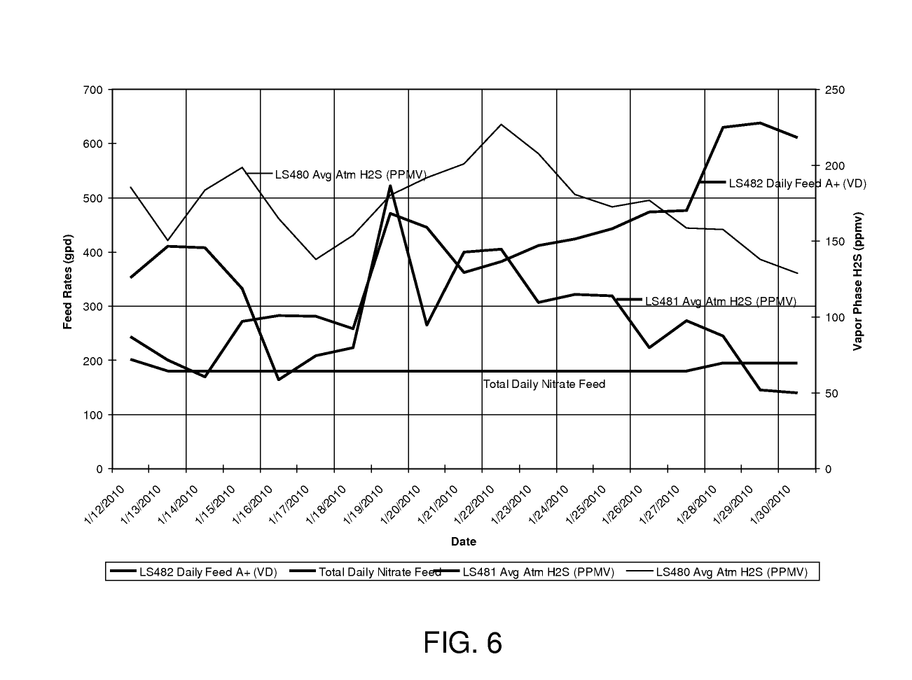

FIG. 7 is a graph showing a six day profile of hydrogen sulfide levels at lift station LS481 of the sewerage system schematically depicted at FIG. 3, utilizing a treatment scheme with the compounds, compositions, systems, and techniques in accordance with one or more aspects of the invention, in FIG. 7, AQUIT refers to the anthraquinone and Bioxide refers to nitrate solution; and

FIG. 8 is a graph showing the hydrogen sulfide levels at lift station LS482 of the sewerage system depicted at FIG. 3, with no treatment and with an addition of a slug dose of anthraquinone (AQUIT).

### DETAILED DESCRIPTION

Some aspects of the invention can involve compounds, compositions, systems, and related techniques that control or reduce objectionable odor characteristics of a body or a stream of wastewater. Some aspects of the invention can involve compounds, compositions, systems, and related techniques that modify or adjust metabolic activity of at least a portion of microorganisms in wastewater to inhibit or disfavor the formation of at least one objectionable odorous compound or species. Some aspects of the invention can involve compounds, compositions, systems, and related techniques that modify, shift, or promote one or more states or characteristics of one or more objectionable odorous species in wastewater. Some aspects of the invention can involve compounds or compositions comprising components that synergistically inhibit, reduce, or control the formation or release of one or more objectionable odorous species in wastewater.

One or more aspects of the compositions, systems, and techniques of the invention can involve compounds that block the generation of sulfide compounds by microorganisms. One or more aspects of the invention can involve utilizing one or more compounds, such as physiochemical modifiers, in compositions, systems, and techniques for controlling odor in wastewater that modify or block at least a portion of a metabolic pathway of microorganisms in the wastewater. One or more aspects of the invention can involve utilizing one or more compounds, compositions, systems and techniques for the control of objectionable odorous species in wastewater, which modify or block a metabolic pathway of sulfur reducing microorganisms in the wastewater. One or more aspects of the invention can involve utilizing one or more compounds in compositions, systems, and techniques for the control of objectionable odorous species in wastewater, which modifies or blocks the reduction of sulfate compounds into sulfide compounds by sulfur reducing microorganisms.

One or more aspects of the invention can involve promoting or enhancing the availability, e.g., bioavailability, of the one or more physiochemical modifiers to disfavor the formation of one or more objectionable metabolites. One or more aspects of the invention can involve providing biofavorable conditions in wastewater that inhibits the metabolic reduction of the sulfate compounds. One or more aspects of the invention can involve enhancing the bioavailability of the one or more physiochemical modifiers by increasing the solubility of such physiochemical modifiers in the wastewater. One or more aspects of the invention can involve the use of compounds, e.g., bioavailability promoter compounds, in compositions, systems, and related methods of odor control.

One or more aspects of the invention can involve shifting or adjusting an equilibrium condition of one or more target odorous species in the wastewater. One or more aspects of the invention can involve disfavoring the formation of one or more objectionable odorous species by adjusting an equilibrium condition of the reaction formation of such species. One or more aspects of the invention can involve compounds in compositions, systems, and related techniques that adjust such reaction conditions of the odorous species. One or more aspects of the invention can involve compounds in compositions that synergistically promote the bioavailability of the one or more physiochemical modifiers while adjusting or shifting the formation conditions of the one or more target odorous species. One or more aspects of the invention can involve compounds in compositions, systems, and related methods that elevate the pH of the wastewater, such as pH-elevating compounds.

One or more aspects of the invention can relate to a method of controlling odor in a sewerage system. The method can involve adding one or more of metabolic or physiochemical modifiers to at least a portion of the wastewater. The method can involve adding one or more pH-elevating compounds to at least a portion of the wastewater. In some embodiments of the invention, the method can involve adding at least one pH-elevating compound to the wastewater to raise the pH thereof to be in a target pH range or target pH value. The target pH range can be a pH value of at least about 8 units, but in some cases, the pH ranges from about 8.2 to about 8.6, and in some cases, a nominal target pH value of about 8.4 units, or at least 8.4 units. The method can comprise adding a composition to wastewater in the sewerage system. The composition typically comprises at least one physiochemical modifiers and at least one bioavailability promoter compounds. In some embodiments of the invention, the physiochemical modifier can comprise at least one anthraquinone and the bioavailability promoter compound can comprise at least one alkaline compound. The composition, in some embodiments of the invention can comprise an alkaline compound and an anthraquinone. In one or more embodiments that can pertain to one or more aspects of the invention, the alkaline compound can be at least one hydroxide selected from the group consisting of alkali hydroxides, alkaline earth hydroxides, alkali earth oxides, and ammonium hydroxides. In one or more other embodiments that can pertain to one or more aspects of the invention, the anthraquinone can be 9,10-anthraquinone and, if appropriate, the alkaline compound can be at least one of sodium hydroxide, potassium hydroxide, calcium hydroxide, and magnesium hydroxide. In some embodiments related to some aspects of the invention, the anthraquinone can be at least one of a haloanthraquinone, an aminoanthraquinone, a hydroxyanthraquinone, and a nitroanthraquinone. One or more further embodiments related to some aspects of the invention can involve adding the composition to the wastewater in an amount sufficient to raise the pH of at least a portion of the wastewater to be in a range that is at least about 8 units. One or more still further embodiments related to some aspects of the invention can involve adding the composition to the wastewater in an amount sufficient to raise the pH of the at least a portion of the wastewater to be in a range of from about 8.2 to about 8.6. One or more further embodiments related to some aspects of the invention can involve adjusting a ratio of an amount of alkaline compound to an amount of the anthraquinone.

One or more aspects of the invention can relate to a wastewater stream comprising an odor controlling composition consisting essentially of a physiochemical modifier and a bioavailability promoter. One or more aspects of the invention can relate to a wastewater stream comprising an odor controlling composition consisting essentially of a physiochemical modifier and an equilibrium shifting compound. One or more aspects of the invention can relate to a wastewater stream comprising an odor controlling composition consisting essentially of an alkaline compound and an anthraquinone. In some embodiments of the wastewater stream, the alkaline compound can be at least one hydroxide selected from the group consisting of alkali hydroxides, alkaline earth hydroxides, alkali earth oxides, and ammonium hydroxides. In some embodiments of the wastewater stream of the invention, the anthraquinone can be at least one of 1,2-anthraquinone, 1,4-anthraquinone, and 2,6-anthraquinone, and 9,10-anthraquinone, 1-nitroanthraquinone, 1-chloroanthraquinone, 1-aminoanthraquinone, 1-hydroxyanthraquinone, 2-hydroxyanthraquinone, 2-aminoanthraquinone, 2-chloroanthraquinone, 1,5-dihydroxyanthraquinone, 2,6-dihydroxyanthraquinone, 1,8-dihydroxyanthraquinone, and 1,4-diaminoanthraquinone.

One or more aspects of the invention method of facilitating odor control in a sewerage system. The method can comprise determining the presence of at least one odorous compound in the sewerage system, and providing an odor control composition consisting essentially of at least one alkaline compound and at least one physiochemical modifier. The method, in accordance with some embodiments for one or more aspects of the invention, can further comprise providing instructions to adjust the relative ratio of an amount of the at least one alkaline compound to an amount of the at least one anthraquinone.

One or more embodiments of the invention can be directed to a system that comprises at least one source of a treating composition having at least one physiochemical modifier and at least one bioavailability promoter or pH-elevating compound. One or more further aspects of the invention can involve one or more sensors or monitoring devices disposed to measure a parameter or condition of the wastewater or one or more components of the odor control system. Non-limiting examples of sensors include composition analyzers, pH sensors, temperature sensors, and flow sensors. One or more further aspects of the invention can involve one or more sensors that provide a signal or representation of the measured parameter of the wastewater. One or more aspects of the invention can involve a control system disposed or configured to receive one or more signal from one or more sensors in an odor control system. The control system can be further configured to provide one or more output or control signals to one the one or more sources of compositions that can comprise, consist essentially of, or consist of one or more physiochemical modifiers and one or more pH-elevating compounds or bioavailability promoters.

The one or more control systems can be implemented using one or more computer systems. The computer system may be, for example, a general-purpose computer such as those based on an Intel PENTIUM®-type processor, a Motorola PowerPC® processor, a Sun UltraSPARC® processor, a Hewlett-Packard PA-RISC® processor, or any other type of processor or combinations thereof. Alternatively, the computer system may include PLCs, specially-programmed, special-purpose hardware, for example, an application-specific integrated circuit (ASIC) or controllers intended for analytical systems.

The control system can include one or more processors typically connected to one or more memory devices, which can comprise, for example, any one or more of a disk drive memory, a flash memory device, a RAM memory device, or other device for storing data. The one or more memory devices can be used for storing programs and data during operation of the odor control system and/or the control subsystem. For example, the memory device may be used for storing historical data relating to the parameters over a period of time, as well as operating data. Software, including programming code that implements embodiments of the invention, can be stored on a computer readable and/or writeable nonvolatile recording medium, and then typically copied into the one or more memory devices wherein it can then be executed by the one or more processors. Such programming code may be written in any of a plurality of programming languages, for example, ladder logic, Java, Visual Basic, C, C#, or C++, Fortran, Pascal, Eiffel, Basic, COBOL, or any of a variety of combinations thereof.

Components of control system may be coupled by one or more interconnection mechanisms, which may include one or more busses, e.g., between components that are integrated within a same device, and/or one or more networks, e.g., between components that reside on separate discrete devices. The interconnection mechanism typically enables communications, e.g., data, instructions, to be exchanged between components of the system.

The control system can further include one or more input devices, for example, a keyboard, mouse, trackball, microphone, touch screen, and one or more output devices, for example, a printing device, display screen, or speaker. In addition, the control system may contain one or more interfaces that can connect to a communication network, in addition or as an alternative to the network that may be formed by one or more of the components of the control system.

According to one or more embodiments of the invention, the one or more input devices may include the one or more sensors for measuring the one or more parameters of the wastewater. Alternatively, the sensors, the metering valves and/or pumps, or all of these components may be connected to a communication network that is operatively coupled to the control system. For example, sensors may be configured as input devices that are directly connected to control system and metering valves and/or pumps of the one or more sources of treating compositions may be configured as output devices that are connected to the control system, and any one or more of the above may be coupled to another ancillary computer system or component so as to communicate with the control system over a communication network. Such a configuration permits one sensor to be located at a significant distance from another sensor or allow any sensor to be located at a significant distance from any subsystem and/or the controller, while still providing data therebetween.

The control system can include one or more computer storage media such as readable and/or writeable nonvolatile recording medium in which signals can be stored that define a program to be executed by one or more processors. The storage or recording medium may, for example, be a disk or flash memory. In typical operation, the processor can cause data, such as code that implements one or more embodiments of the invention, to be read from the storage medium into a memory device that allows for faster access to the information by the one or more processors. The memory device is typically a volatile, random access memory such as a dynamic random access memory (DRAM) or static memory (SRAM) or other suitable devices that facilitates information transfer to and from the one or more processors.

Although the control system is described by way of example as one type of computer system upon which various aspects of the invention may be practiced, it should be appreciated that the invention is not limited to being implemented in software, or on the computer system as exemplarily shown. Indeed, rather than implemented on, for example, a general purpose computer system, the controller, or components or subsections thereof, may alternatively be implemented as a dedicated system or as a dedicated programmable logic controller (PLC) or in a distributed control system. Further, it should be appreciated that one or more features or aspects of the invention may be implemented in software, hardware or firmware, or any combination thereof. For example, one or more segments of an algorithm executable by the one or more controllers can be performed in separate computers, which in turn, can be communication through one or more networks.

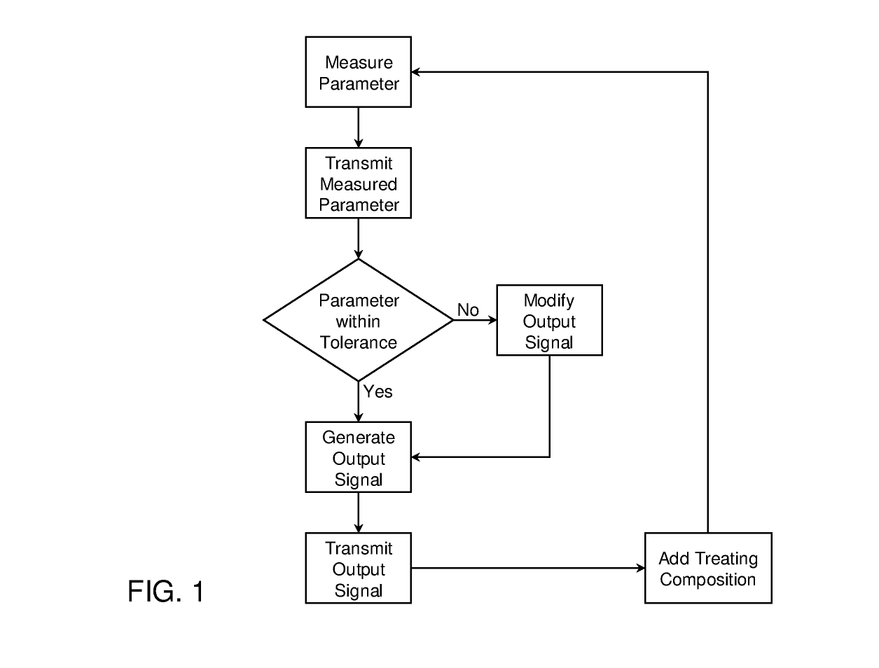

FIG. 1 is an exemplary flowchart that depicts an exemplary algorithm in one or more control systems and techniques in accordance with one or more aspects of the invention. The control approach can involve measuring one or more parameters or conditions of the odor control system, wastewater in the sewerage system, and/or an environment of the sewerage system such as the headspace in a sewerage line. Control can then comprise transmitting the measured parameter and determining if the measured parameter is within tolerance of a target value of the parameter. The parameter can be, for example, the pH of the wastewater, the concentration of an odorous species, or both. The tolerance can be, for example, within 10% of the target value or, in some configurations, within 5% of the target value. If the measured parameter is not within the tolerance, then an output signal is modified, generated, and transmitted to a source of treating composition comprising, consisting essentially of, or consisting of one or more anthraquinone compounds and one or more alkaline compounds. The control system can be implemented to involve separate control algorithms for each of the physiochemical modifier and the pH elevating or bioavailability promoter. If the measured parameter is within the tolerance condition, then the output signal is optionally generated and transmitted to the source of the treating composition, which can be at least one anthraquinone, at least one alkaline compound, alone or as a mixed composition of both. The depicted closed loop control scheme is exemplarily presented in a feedback loop but one or more aspects of the invention can be implemented utilizing a feedforward control approach.

The one or more treating compositions, having at least one anthraquinone, at least one alkaline compound, alone or in a mixed composition, may be introduced into a wastewater stream in a sewerage system at a first location. The one or more sensors may be disposed at the point of introduction, downstream of the point of introduction, or upstream of the point of introduction.

Further, an open control scheme may also be utilized, alone or with closed loop control scheme. For example, a predetermined treating schedule may be utilized. The predetermined treating schedule may utilize a plurality of time-of-day, day-of-week, and/or month-of-year target treating output values. For example, the treating schedule may comprise an array of control values that varies hourly, daily, and/or monthly.

### EXAMPLES

The function and advantages of these and other embodiments of the invention can be further understood from the examples below, which illustrate the benefits and/or advantages of the one or more systems and techniques of the invention but do not exemplify the full scope of the invention.

### Example 1

This example describes a novel approach to odor control that utilized pH adjustment and nitrate addition in a sewage collection system which realized a 42% cost reduction as compared with the use of nitrate salts alone. Atmospheric hydrogen sulfide and dissolved sulfide concentrations were controlled to the same levels with the new approach as with the nitrate throughout the system.

The addition of an anthraquinone to the alkaline material used for pH adjustment further resulted in an unexpected 21% decrease in atmospheric hydrogen sulfide concentration at the downstream monitoring point and a drop in dissolved sulfide from 0.2 to 0.0 ppmv at the plant.

The combination of nitrate and pH shift processes provided odor control and the addition of anthraquinone further reduces odor and corrosion in wastewater collection systems beyond the expected level.

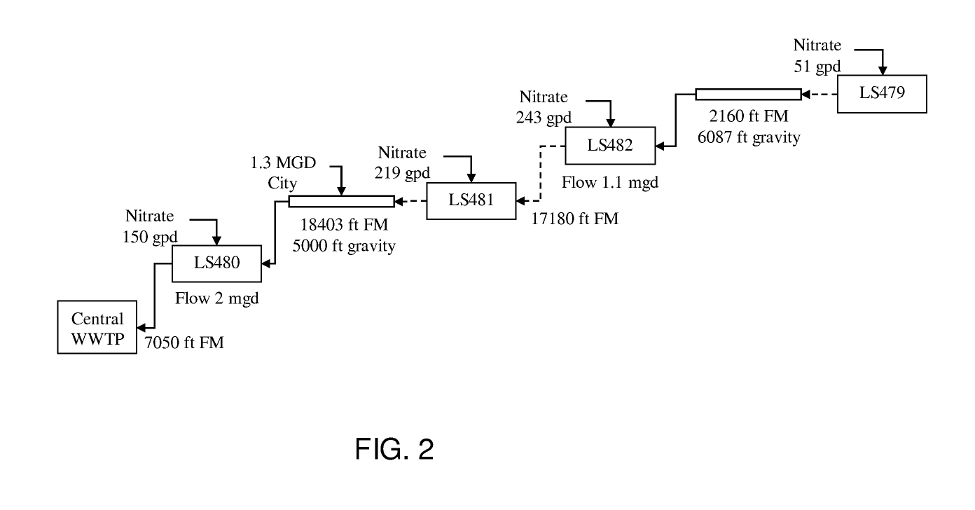

An existing sewerage collection system with a series of lift stations originating along a major thoroughfare was selected as the study site for odor control chemistry utilizing calcium hydroxide, nitrate salts, and anthraquinone. The collection system consisted of four serial master lift stations LS 479, LS482, LS 481, and LS 480 feeding wastewater to a central treatment plant WWTP as depicted in FIG. 2. Historically odors in the collection system have been controlled by the addition of nitrate salts only.

Lift station LS 479 was fed by gravity lines. During the period from June 23 to July 14, a nitrate salt solution was added into this lift station at an average of about 51.4 gallons per day (GPD).

The force main from LS479 traveled about 2,160 feet to manhole where it continued to a gravity line for about 6,087 feet to terminate at a manhole about 50 feet north of lift station LS482. During July, flow through lift station LS482 averaged about 1.1 MGD. During the period from June 23 to Jul. 14, 2009, nitrate salt feed into LS482 averaged about 243 GPD.

The force main from LS482 traveled about 17,180 feet to a manhole about 50 feet south of lift station LS481. This manhole served as one of the monitoring points for the chemical feed at LS482. Retention time in the line averaged about 9 hours. During the period from June 23 to July 14, nitrate salt solution that was added into lift station LS481 averaged about 219 GPD.

The force main from lift station LS481 proceeded west, then south, and west again about 100 feet to another manhole. The total force main distance was about 18,304 feet. At this latter manhole, the wastewater flow was combined with approximately 1.3 MGD from the city, which doubles the wastewater flow.

Wastewater then flowed from lift station LS481 to lift station LS480, which served as a monitoring point for an upstream chemical feed. The estimated total flow through lift station LS480 was about 2 MGD. During the period from June 23 to July 14, nitrate salt solution feed into lift station LS481 averaged about 150 GPD.

The force main from lift station LS480 traveled about 7,050 feet west to the city's treatment plant WWTP where a tap in the line was used as the final monitoring point for dissolved sulfide. For odor control, the dissolved sulfide target level was less than 1 ppm at this point.

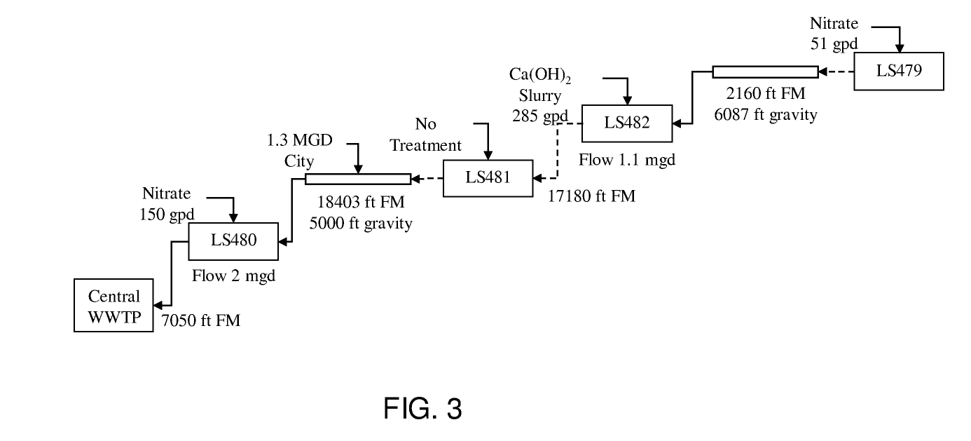

FIG. 3 shows the proposed treatment scheme. Calcium hydroxide (with or without anthraquinone) was to be added at lift station LS 482 to control hydrogen sulfide emission at the lift station and downstream. Calcium hydroxide (with or without anthraquinone) feed rate was dependent mainly on the wastewater flow rate.

Table 1 summarizes the treatment quantities by lift station using nitrate salt. Table 2 summarizes the estimated feed rates anticipated prior to actual deployment. The anticipated materials cost saving would be between 10 and 20 percent.

TABLE 1
Comparison Treatment Summary

| | Dose Rate |
|---|---|
| | Nitrate Salt |
| Lift Station | Solution (GPD) |
| 479 | 51 |
| 482 | 243 |
| 481 | 236 |
| 480 | 111 |
| Total | 641 |

TABLE 2
Proposed Treatment Summary

| Lift Station | Product | Dose Rate (GPD) |
|---|---|---|
| LS479 | Nitrate Salt Solution | 51 |
| LS482 | Calcium Hydroxide Slurry | 285 |
| LS481 | None | — |
| LS480 | Nitrate Salt Solution | 150 |

Baseline data was collected while adding nitrate salt solution at the four lift stations at the noted feed rates during the period from June 23 to July 14. Data collected included atmospheric hydrogen sulfide collected every five minutes with monitor/loggers within the monitoring manhole at lift station LS481 and inside the lift station LS480, and dissolved sulfide grab samples at each as well as treatment plant WWTP. Nitrate residual and pH data were also collected. During the baseline period, the calcium hydroxide storage and feed system was constructed and installed on the LS482 site, which consisted of a 6150 gallon storage tank, mixing system, peristaltic pump, VersaDose™ controller, and a pH monitor. The chemical feed line was disposed to feed into the manhole about 50 feet upstream of lift station LS482.

Calcium hydroxide slurry was delivered to the site on July 14 and added on a dosing curve. Nitrate salt solution feed was terminated at lift stations LS482 and LS481. Dosing curve feed of the calcium hydroxide slurry continued until August 4 when the feed control was changed to be driven by the pH of the sewage entering the lift station. Over the next few weeks the controller pH set point was adjusted until the desired atmospheric pH was attained downstream at lift station LS481.

Once the pH set point was established and the required calcium hydroxide slurry feed was determined, a slug of ten gallons of 50% anthraquinone was added at the manhole to determine the effect of adding anthraquinone in concert with calcium hydroxide.

Two batches of a formulation of calcium hydroxide supplemented with anthraquinone were fed to determine the effectiveness of the combination for controlling odor.

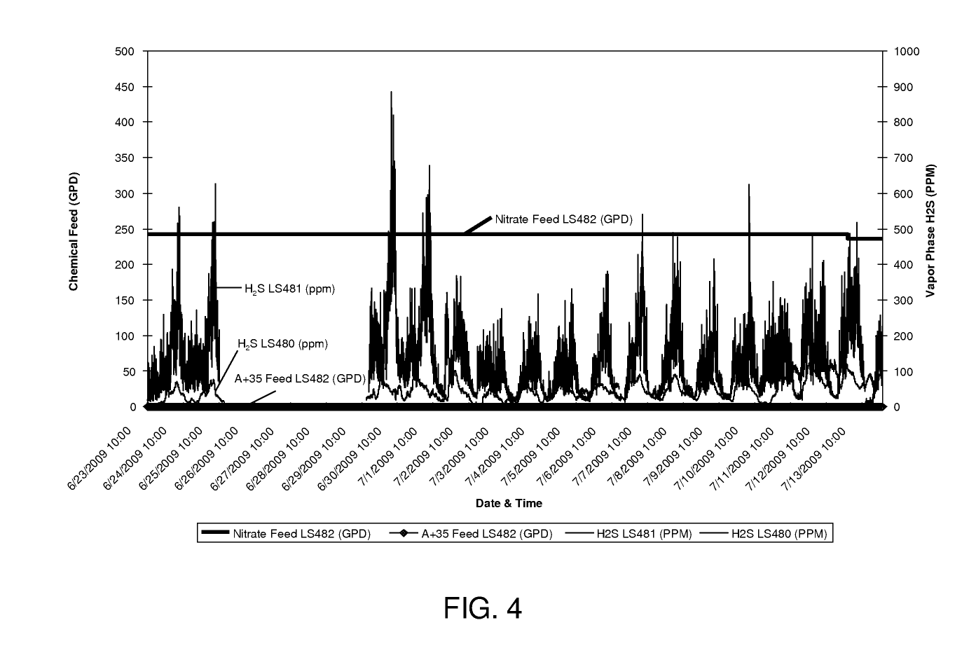

The primary monitoring point for atmospheric hydrogen sulfide was at lift station LS481. The primary monitoring point for dissolved sulfide was the plant influent. During the period from June 23 to July 14 background data was gathered (FIG. 4) to reflect the system operating on nitrate salt feed at all four lift stations. Tables 3-9 summarize the collected data.

TABLE 3
Background Data Summary

| | Nitrate | Calcium | LS481 | LS481 | LS480 | LS480 | |
|---|---|---|---|---|---|---|---|
| | Salt | Hydroxide | S²⁻ MH | Avg Atm | S²⁻ WW | Avg Atm | WTP S²⁻ |
| | Solution | Slurry Feed | In Grab | H₂S | In Grab | H₂S | In Grab |
| Date | Feed (GPD) | (GPD) | (mg/L) | (ppmv) | (mg/L) | (ppmv) | (mg/L) |
| 6/23-7/13 | 641 | 0 | 1.6 | 131 | 2.2 | 66 | 1.3 |

The average hydrogen sulfide at lift station LS480 during this comparison period was 131 ppmv with a standard deviation of 50 ppmv.

Tables 4-9 summarize performance data at control or monitoring points.

TABLE 4
Summary Data for period 7/15 to 7/31

| | Nitrate | Calcium | LS481 | LS481 | LS480 | LS480 | |
|---|---|---|---|---|---|---|---|
| | Salt | Hydroxide | S²⁻ MH | Avg Atm | S²⁻ WW | Avg Atm | WTP S²⁻ |
| | Solution | Slurry Feed | In Grab | H₂S | In Grab | H₂S | In Grab |
| Date | Feed (GPD) | (GPD) | (mg/L) | (ppmv) | (mg/L) | (ppmv) | (mg/L) |
| 7/15 to 7/31 | 187 | 211 | 6.0 | 242 | 6.3 | 206 | 1.3 |

Calcium hydroxide slurry feed (A+) was based on a fixed curve at LS482

TABLE 5
Summary Data from period 08/01 to 08/03

| | Nitrate | Calcium | LS481 | LS481 | LS480 | LS480 | |
|---|---|---|---|---|---|---|---|
| | Salt | Hydroxide | S²⁻ MH | Avg Atm | S²⁻ WW | Avg Atm | WTP S²⁻ |
| | Solution | Slurry Feed | In Grab | H₂S | In Grab | H₂S | In Grab |
| Date | Feed (GPD) | (GPD) | (mg/L) | (ppmv) | (mg/L) | (ppmv) | (mg/L) |
| 8/1-8/3 | 185 | 23 | ND | 435+* | ND | 231 | ND |

*Value is low because sensor was found to be maxed out at 1,000 ppm several times during the logging session.

TABLE 6
Summary Data for period 08/04 to 09/14

| | Nitrate | Calcium | LS481 | LS481 | LS480 | LS480 | |
|---|---|---|---|---|---|---|---|
| | Salt | Hydroxide | S²⁻ MH | Avg Atm | S²⁻ WW | Avg Atm | WTP S²⁻ |
| | Solution | Slurry Feed | In Grab | H₂S | In Grab | H₂S | In Grab |
| Date | Feed (GPD) | (GPD) | (mg/L) | (ppmv) | (mg/L) | (ppmv) | (mg/L) |
| 8/4 to 8/10 | 204 | 192 | 4.0 | 195 | 6.0 | 226 | 2.8 |
| 8/12 to 8/14 | 198 | 234 | 2.7 | 204 | 3.4 | 187 | 3.4 |
| 8/15 to 8/17 | 197 | 254 | ND | 178 | ND | 172 | ND |
| 8/19 to 9/14 | 202 | 246 | 3.0 | 146 | 5.1 | 165 | 0.5 |

Comparison of atmospheric hydrogen sulfide at LS480 before calcium hydroxide slurry feed and during calcium hydroxide slurry feed is invalid since the lift station was ventilated at the beginning of the trial, then intermittently turned off.

Table 3 above lists the baseline nitrate salt feed and downstream sulfide data. A performance summary was prepared using a composite of all values using the initial formulation of the calcium hydroxide slurry. Table 7 lists the composite summary.

TABLE 7
Composite Summary of Performance

| | Nitrate | Calcium | LS481 | LS481 | LS480 | LS480 | |
|---|---|---|---|---|---|---|---|
| | Salt | Hydroxide | S²⁻ MH | Avg Atm | S²⁻ WW | Avg Atm | WTP S²⁻ |
| | Solution | Slurry Feed | In Grab | H₂S | In Grab | H₂S | In Grab |
| Date | Feed (GPD) | (GPD) | (mg/L) | (ppmv) | (mg/L) | (ppmv) | (mg/L) |
| 7/13 to 10/17 | 198 | 221 | 3.4 | 134 | 6.0 | 210 | 2.0 |

Table 7 is a composite of values taken for period 7/13 to 10/17. Table 7 includes days in which nitrate salt solution feed at lift stations LS479 and LS480 were operating and calcium hydroxide slurry feed at lift station LS482 was operating.

To test the effect in an alkaline enhanced sewer, a ten gallon slug dose of the anthraquinone was added at lift station LS482, and the results downstream are presented in Table 8.

TABLE 8
Comparison of Downstream Sulfides Prior to and After Anthraquinone Slug Dose.

| | | | | | | | |
|---|---|---|---|---|---|---|---|
| | Nitrate | Calcium | LS481 | LS481 | LS480 | LS480 | |
| | Salt | Hydroxide | S²⁻ MH | Avg Atm | S²⁻ WW | Avg Atm | WTP S²⁻ |
| | Solution | Slurry Feed | In Grab | H₂S | In Grab | H₂S | In Grab |
| Date | Feed (GPD) | (GPD) | (mg/L) | (ppmv) | (mg/L) | (ppmv) | (mg/L) |

10/4 to 10/13 Nitrate salt solution feed at LS479 & LS480,
Dose curve for calcium hydroxide slurry feed at LS482.

| | | | | | | | |
|---|---|---|---|---|---|---|---|
| 10/4 to 10/13 | 194 | 292 | 5.2 | 160 | 7.6 | 400 | 1.4 |

10/14 to 10/17 Nitrate salt solution feed at lift stations LS479 and LS480, Dose curve for
calcium hydroxide slurry feed at lift station LS482. 10 gal Anthraquinone was added on 10/13.

| | | | | | | | |
|---|---|---|---|---|---|---|---|
| 10/14 to 10/17 | 197 | 258 | 0 | 100 | ND | 305 | ND |

On 10/21, calcium hydroxide slurry feed was interrupted and was resumed on 12/04; the feed rate was increased, and feed was continued on dosing curve for 3 days.

TABLE 9
November/December Calcium Hydroxide Slurry Feed Summary.

| | | | | | | | |
|---|---|---|---|---|---|---|---|
| | Nitrate | Calcium | LS481 | LS481 | LS480 | LS480 | |
| | Salt | Hydroxide | S²⁻ MH | Avg Atm | S²⁻ WW | Avg Atm | WTP S²⁻ |
| | Solution | Slurry Feed | In Grab | H₂S | In Grab | H₂S | In Grab |
| Date | Feed (GPD) | (GPD) | (mg/L) | (ppmv) | (mg/L) | (ppmv) | (mg/L) |

11/20 to 12/3: Nitrate salt solution feed at LS479 & LS480,
Curve control calcium hydroxide slurry feed at LS482.

| | | | | | | | |
|---|---|---|---|---|---|---|---|
| 11/20 to 12/3 | 197 | 320 | 20 | 141 | 8 | 193 | 8 |

12/5 to 12/7: Nitrate salt solution feed at LS479 & LS480,
Curve control calcium hydroxide slurry feed at LS482.

| | | | | | | | |
|---|---|---|---|---|---|---|---|
| 12/5 to 12/7 | 188 | 439 | ND | 86 | ND | 172 | ND |

12/9 to 12/19 Nitrate salt solution feed at LS479 & LS480,
pH 8.5-8.8 control calcium hydroxide slurry feed at LS482.

| | | | | | | | |
|---|---|---|---|---|---|---|---|
| 12/9 to 12/19 | 183 | 335 | 3.0 | 199 | 5 | 163 | 0.1 |

The system was shut down during the winter holiday and then resumed in early January providing the data summary in Table 10.

TABLE 10
January Calcium Hydroxide Slurry Feed Summary

| | | | | | | | |
|---|---|---|---|---|---|---|---|
| | Nitrate | Calcium | LS481 | LS481 | LS480 | LS480 | |
| | Salt | Hydroxide | S²⁻ MH | Avg Atm | S²⁻ WW | Avg Atm | WTP S²⁻ |
| | Solution | Slurry Feed | In Grab | H₂S | In Grab | H₂S | In Grab |
| Date | Feed (GPD) | (GPD) | (mg/L) | (ppmv) | (mg/L) | (ppmv) | (mg/L) |

1/12 10 1/30: Nitrate salt solution feed at lift stations LS479 and LS480.
pH controlled calcium hydroxide feed - drifting.

| | | | | | | | |
|---|---|---|---|---|---|---|---|
| 1/12 to 1/30 | 184 | 394 | 6.2 | 107 ± 37 | 8.9 | 174 ± 26 | 1 |

Calcium hydroxide slurry feed was continued with dosing curve control changing only the global factor as noted below until 02/22, when the feed material was converted from calcium hydroxide slurry to calcium hydroxide/anthraquinone blend.

TABLE 11
February Calcium Hydroxide Slurry Feed Summary

| | | | | | | | |
|---|---|---|---|---|---|---|---|
| | Nitrate | Calcium | LS481 | LS481 | LS480 | LS480 | |
| | Salt | Hydroxide | S²⁻ MH | Avg Atm | S²⁻ WW | Avg Atm | WTP S²⁻ |
| | Solution | Slurry Feed | In Grab | H₂S | In Grab | H₂S | In Grab |
| Date | Feed (GPD) | (GPD) | (mg/L) | (ppmv) | (mg/L) | (ppmv) | (mg/L) |

2/10: Nitrate salt solution feed at LS479 and LS480.
Calcium hydroxide slurry curve dose at 100%.

| | | | | | | | |
|---|---|---|---|---|---|---|---|
| 2/9 to 2/10 | 188 | 552 | ND | 40 | ND | 121 | ND |

2/10 to 2/11: Nitrate salt solution feed at LS479 & LS480,
calcium hydroxide slurry curve dose at 80%.

| | | | | | | | |
|---|---|---|---|---|---|---|---|
| 2/10 to 2/11 | 188 | 326 | ND | 69 | ND | 119 | ND |

2/11 to 2/15: Nitrate salt solution feed at LS479 & LS480,
calcium hydroxide slurry curve dose at 70%

| | | | | | | | |
|---|---|---|---|---|---|---|---|
| 2/11 to 2/15 | 188 | 281 | ND | 152 | ND | 197 | ND |

2/15 to 2/17 Nitrate salt solution feed at LS479 & LS480,
calcium hydroxide slurry curve dose at 75%

| | | | | | | | |
|---|---|---|---|---|---|---|---|
| 2/15 to 2/17 | 188 | 308 | ND | 110 | ND | 208 | ND |

2/17 to 2/22 Nitrate salt solution feed at LS479 & LS480,
calcium hydroxide slurry curve dose at 72%

| | | | | | | | |
|---|---|---|---|---|---|---|---|
| 2/17 to 2/22 | 188 | 308 | 6.5 | 110 | 9.2 | 216 | 2.1 |

3/6 to 3/11 Nitrate salt solution feed at LS479 & LS480,
Calcium hydroxide/anthraquinone curve dose at 72%

| | | | | | | | |
|---|---|---|---|---|---|---|---|
| 3/6 to 3/11 | 182 | 318 | 6.0 | 100 | 13 | 277 | 1 |

The data was tabulated for every day on which no calcium hydroxide was fed and that nitrate salt was fed at all four lift stations. Data was also tabulated for all days that nitrate salt was off at lift stations LS482 and LS481 and calcium hydroxide was fed at lift station LS482 and the average hydrogen sulfide at lift station LS481 for the day was within one half standard deviation of the value when nitrate salt was fed at all stations.

TABLE 12
Trial Average Feed Rate Summary

| | Daily Feed of | Total Daily | | |
|---|---|---|---|---|
| | Calcium | Feed of Nitrate | | |
| | Hydroxide | Salt Solution in | LS481 | WWTP |
| | Slurry at LS482 | the System | Avg Atm | S²⁻ In |
| | (GPD) | (GPD) | H₂S (ppmv) | Grab (mg/L) |
| Average | 0 | 627 | 129 | 0.93 |
| Average | 303 | 180 | 130 | 0.83 |

No flow data for any of the lift stations except for LS480 and the data provided were monthly average daily flows as follows.

TABLE 13
Wastewater Flow Rate Summary

| | Avg Flow |
|---|---|
| Month | (MGD) |
| June 2009 | 2.341 |
| July 2009 | 2.113 |
| August 2009 | 2.566 |
| September 2009 | 3.403 |
| October 2009 | 3.224 |
| November 2009 | 3.132 |
| December 2009 | 2.872 |
| January 2010 | 1.494 |
| February 2010 | 1.834 |
| March 2010 | 1.814* |

*03/01 to 03/10

A secondary objective for the trial is the test of a product blended with calcium hydroxide to improve results. Anthraquinone was proposed for this formulation.

As noted above, the flow through lift station LS480 was not significantly different in March than in February, and so the effect of flow difference is avoided by comparing data for those two months for feed of calcium hydroxide and calcium hydroxide/anthraquinone blend. This chart reflects days that hydrogen sulfide concentrations were within target range.

TABLE 14
Initial Calcium Hydroxide - Calcium Hydroxide/Anthraquinone
Blend Comparison

| | Daily Feed of | | | |
|---|---|---|---|---|
| | Calcium | | | |
| | Hydroxide | Total | | |
| | or Calcium | Daily Feed | | |
| | Hydroxide/ | of Nitrate | LS481 | WWTP |
| | Anthraquinone | Salt | Avg Atm | S²⁻ In |
| | Blend at | Solution | H₂S | Grab |
| | LS482 (GPD) | (gal) | (ppmv) | (mg/L) |
| 2/1 to 2/22 Calcium | 319 | 190 | 127 | 1 |
| Hydroxide Slurry | | | | |
| Average | | | | |
| 3/6 to 3/11 Calcium | 318 | 183 | 100 | 1 |
| Hydroxide/ | | | | |
| Anthraquinone | | | | |
| Slurry Average | | | | |

A similar trial was repeated in May. Summary results are presented in Table 15.

TABLE 15
Second Calcium Hydroxide Without and With Anthraquinone Comparison

| | Daily Feed of | | | |
|---|---|---|---|---|
| | Calcium | Total | | |
| | Hydroxide | Daily Feed | | |
| | or Calcium | of Nitrate | LS481 | WWTP |
| | Hydroxide/ | Salt | Avg Atm | S²⁻ In |
| | Anthraquinone | Solution | H₂S | Grab |
| | at LS482 (gal) | (gal) | (ppmv) | (mg/L) |
| 5/7-5/10 Calcium | 327 | 194 | 238 | 0.2 |
| Hydroxide Slurry | | | | |
| Average | | | | |
| 5/12-5/14 Calcium | 308 | 194 | 186 | 0.0 |
| Hydroxide/ | | | | |
| Anthraquinone | | | | |
| Slurry Average | | | | |

Data was taken over a six month period to test the validity and performance of the addition of calcium hydroxide slurry, and a blend of calcium hydroxide and anthraquinone for odor and corrosion control.

A slurry of calcium hydroxide was used.

The data shows that maintaining atmospheric hydrogen sulfide to levels that observed when nitrate salts were fed throughout the system, maintaining dissolved sulfide concentration of 1 mg/L or less in the treatment plant influent, and reducing the treatment cost for the utility were achieved. By raising the pH of the sewage, sulfide was retained in a nonvolatile state and was not released into the atmosphere in the collection system. By keeping the sulfides in solution as generated, the nitrate could be utilized for sulfide removal rather that sulfide prevention, a far more efficient process. Finally, since the sulfide removal was taking place with additional alkalinity, the reaction was more efficient. Thus the combination of additives lowered the cost of treatment.

Nitrate salt is added to the sewage at lift station LS480 for removal of reduced sulfur by oxidation to meet the goal of less than 1 ppm in the plant influent. This enhanced efficiency because of the alkaline material added at lift station 482.

The calcium hydroxide and calcium hydroxide-anthraquinone blend were added into a manhole about 50 feet ahead of the lift station through a reinforced tubing driven by a peristaltic pump controlled by a VersaDose™ system attached to a pH controller.

An attempt was made to remove from consideration those data days when extraordinary events affected the results. Data was removed for days that experienced high rainfall and those immediately following.

The flows varied on a monthly average at lift station LS480 from a low of 1.494 MGD to a high of 3.402 MGD during the study. This demonstrated particular advantages of the present dose to demand feed. The automated PLC-based control system was demonstrated to automatically adjust to the changing flows, ensuring proper treatment without wasteful overfeed.

The data shows that raising the pH with calcium hydroxide in conjunction with nitrate salts can be a viable and cost-effective treatment technique for odor and corrosion control in this wastewater collection system, as shown by the data in Table 16.

Calcium hydroxide with nitrate salt proved to be a more economical treatment approach than nitrate salt only in the trial system. The cost savings to the utility exceeded expectations and were as high as 48%.

Maintaining atmospheric hydrogen sulfide level at lift station LS481 within half a standard deviation of what was experienced treating only with nitrate salt was attained.

TABLE 16
Trial Average Atmospheric Sulfide Summary

| | Daily Feed of | Total Daily | LS481 | LS480 |
|---|---|---|---|---|
| | Calcium | Feed of Nitrate | Avg | Avg |
| | Hydroxide | Salt Solution in | Atm | Atm |
| | Slurry at | the System | H₂S | H₂S |
| | LS482 (GPD) | (GPD) | (ppmv) | (ppmv) |
| Average | 0 | 627 | 129 | 68 |
| Average | 303 | 180 | 130 | 81 |

The dissolved sulfide goal of less than 1 mg/L at plant WWTP was achieved as noted by the data presented at Table 17.

TABLE 17
Trial Average WWTP Dissolved Sulfide

| | Daily Feed of | Total Daily Feed | |
|---|---|---|---|
| | Calcium | of Nitrate Salt | |
| | Hydroxide Slurry | Solution in the | WWTP S²⁻ In |
| | at LS482 (GPD) | System (GPD) | Grab (mg/L) |
| Average | 0 | 627 | 0.93 |
| Average | 303 | 180 | 0.83 |

The data presented in Tables 16 and 17 also shows that the treatment technique of addition of alkaline material and nitrate salt at separate feed points in the collection system successfully attained the treatment goals.

Experience in operating this system has shown that calcium nitrate with anthraquinone is the can be advantageously utilized as a treatment product for odor and corrosion control. The composition can be fed by peristaltic pumps through relatively small diameter tubing while maintaining a high concentration of active ingredient.

Depending on site conditions, the estimated dose rate of calcium hydroxide or calcium hydroxide/anthraquinone slurry is about 100 to about 300 gallons per million gallons of sewage flow.

A one time slug of anthraquinone along with the calcium hydroxide feed provided an about 38% reduction in the hydrogen sulfide concentration at the downstream monitoring point lift station LS481 over the next four days.

The addition of calcium hydroxide (A+) for odor and corrosion control showed improvement in atmospheric hydrogen sulfide concentrations.

A review of treatment costs with various schemes shows (Table 18) that the savings were greater using calcium hydroxide alone. It should be noted that the blend of calcium hydroxide with anthraquinone improved levels for both atmospheric and dissolved sulfide.

TABLE 18
Additive Treatment Savings

| Treatment Scheme | Treatment Cost Savings |
|---|---|
| Nitrate Salt at All Four Lift Stations | — |
| Calcium Hydroxide at lift station LS482, | 43% |
| Nitrate Salt at lift station LS480 | |
| Calcium Hydroxide/Anthraquinone at lift | 41% |
| station LS482, Nitrate Salt at lift station | |
| LS480 | |

### Example 2

This example is an addendum to Example 1 and further evaluates the synergism between an alkaline compound and an anthraquinone in preventing or reducing atmospheric hydrogen sulfide in sewerage systems. The same sewerage system as in Example 1 was utilized in this evaluation.

As noted in Example 1, treating with calcium hydroxide and anthraquinone was more effective that treating with calcium hydroxide alone. This example evaluates the effect of treating with anthraquinone alone, and shows that the effect of treating with a mixture with calcium hydroxide was more effective than the sum of adding each alone.

In order to gather the required information, the two OdaLog® hydrogen sulfide monitor/loggers were deployed in the manhole just prior to lift station LS481 prior to 10:00 a.m. on day one. At 10:00 a.m. on day one all chemical feed was turned off at lift station LS482. At 10:00 a.m. on day two a ten gallon slug of anthraquinone (AQUIT) was added to the flow through the manhole at lift station LS482. At 10:00 a.m. on day three, regular chemical feed was resumed at lift station LS482. The OdaLog® monitor/loggers were retrieved on day six and downloaded to retrieve the atmospheric hydrogen sulfide concentrations before, during, and following the trial.

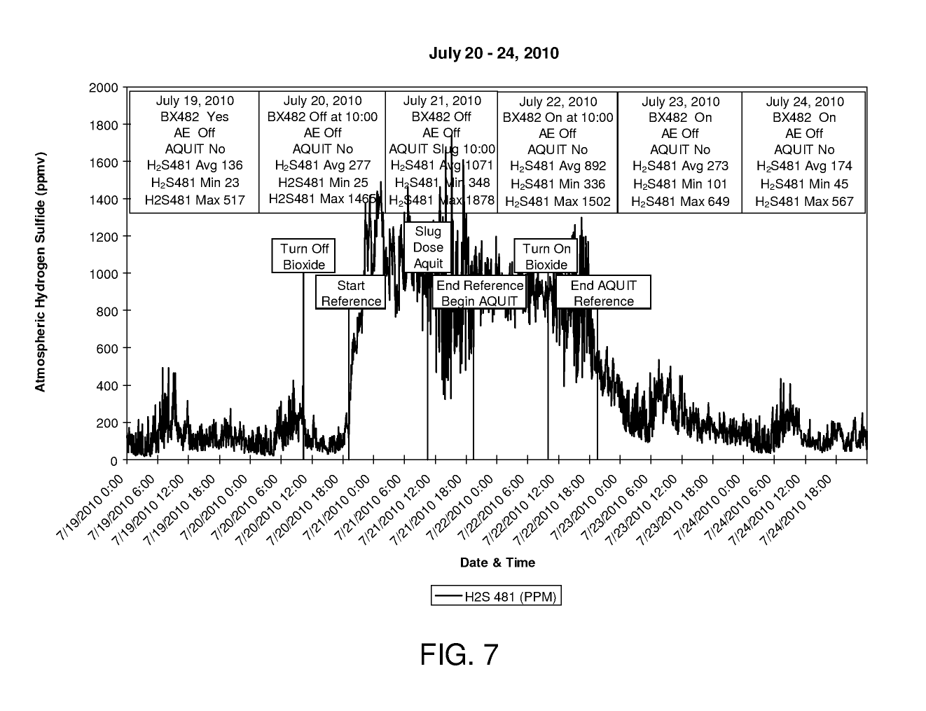

Data was collected over a period of several days to include a full day prior to the test and a full day after the test as summarized in the graph of FIG. 7.

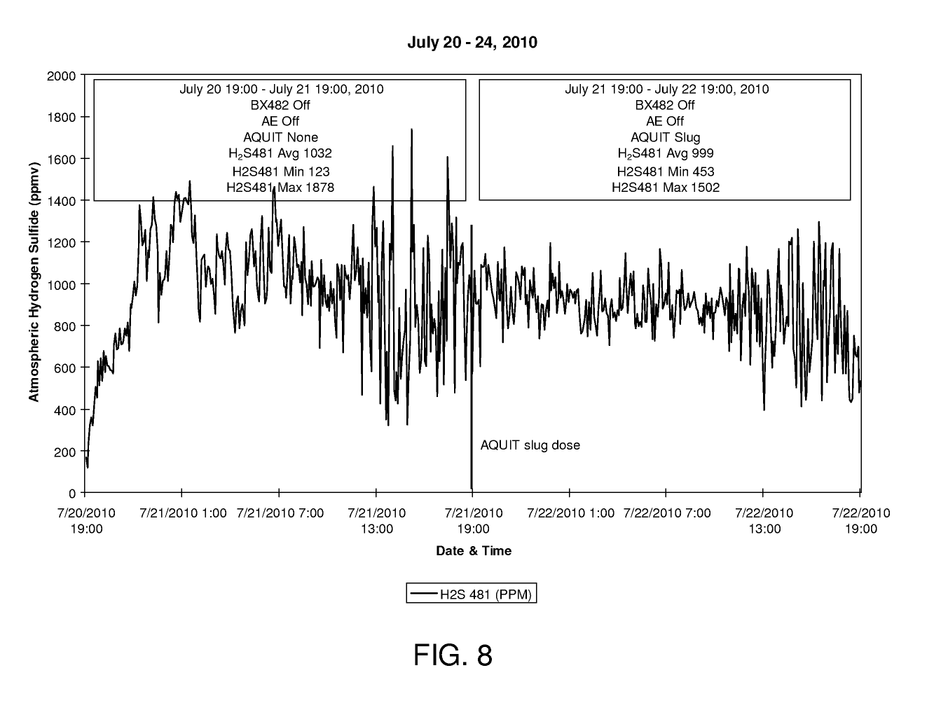

The detention time in the sewer between lift stations LS482 and LS481 was determined to be nine hours, and so the effect of the events at lift station LS482 were seen at lift station LS481 at about nine hours later. The data for the 24 hour period at lift station LS482 starting at time 19:00 is presented in FIG. 8. The average atmospheric hydrogen sulfide concentration for the 24 hour period with no chemical additive was about 1,032 ppmv. During the following 24 hour period during which the effect of the slug dose of anthraquinone, the atmospheric hydrogen sulfide concentration averaged about 999 ppmv; the hydrogen sulfide concentration was thus reduced by about 3.2 percent.

In contrast, when anthraquinone was added with calcium hydroxide to the sewer upstream of the sampling point, the atmospheric hydrogen sulfide downstream dropped 37.5 percent as noted above (see Table 8).

The data thus indicates the synergistic effect of calcium hydroxide and anthraquinone for the prevention, inhibition, and/or removal of atmospheric hydrogen sulfide.

Having now described some illustrative embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives.

Those skilled in the art should appreciate that the parameters and configurations described herein are exemplary and that actual parameters and/or configurations will depend on the specific application in which the systems and techniques of the invention are used. Those skilled in the art should also recognize or be able to ascertain, using no more than routine experimentation, equivalents to the specific embodiments of the invention. It is therefore to be understood that the embodiments described herein are presented by way of example only and that, within the scope of the appended claims and equivalents thereto; the invention may be practiced otherwise than as specifically described.

Moreover, it should also be appreciated that the invention is directed to each feature, system, subsystem, or technique described herein and any combination of two or more features, systems, subsystems, or techniques described herein and any combination of two or more features, systems, subsystems, and/or methods, if such features, systems, subsystems, and techniques are not mutually inconsistent, is considered to be within the scope of the invention as embodied in the claims. Further, acts, elements, and features discussed only in connection with one embodiment are not intended to be excluded from a similar role in other embodiments.

As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A method of controlling odor in a sewerage system, comprising:
adding at least one alkaline compound to a wastewater in the sewerage system; and
adding at least one anthraquinone to the wastewater.

2. The method of claim 1, wherein the alkaline compound is at least one hydroxide selected from the group consisting of alkali hydroxides, alkaline earth hydroxides, alkali earth oxides, and ammonium hydroxides.

3. The method of claim 2, wherein the anthraquinone is 9,10-anthraquinone and the alkaline compound is at least one of sodium hydroxide, potassium hydroxide, calcium hydroxide, and magnesium hydroxide.

4. The method of claim 2, wherein the anthraquinone is at least one of 9,10-anthraquinone, a haloanthraquinone, an aminoanthraquinone, a hydroxyanthraquinone, and a nitroanthraquinone.

5. The method of claim 1, wherein the at least one alkaline compound is added to the wastewater in an amount sufficient to raise the pH of at least a portion of the wastewater to be at least about 8 units.

6. The method of claim 5, wherein the at least one alkaline compound is added to the wastewater in an amount sufficient to raise the pH of the at least a portion of the wastewater to be in a range of from about 8.2 to about 8.6.

7. The method of claim 1, further comprising adjusting a ratio of an amount of alkaline compound to an amount of the anthraquinone.

8. A wastewater stream comprising an odor controlling composition consisting essentially of an alkaline compound and an anthraquinone.

9. The wastewater stream of claim 8, wherein the alkaline compound is at least one hydroxide selected from the group consisting of alkali hydroxides, alkaline earth hydroxides, alkali earth oxides, and ammonium hydroxides.

10. The wastewater stream of claim 8, wherein the anthraquinone is at least one of 1,2-anthraquinone, 1,4-anthraquinone, and 2,6-anthraquinone, and 9,10-anthraquinone, 1-nitroanthraquinone, 1-chloroanthraquinone, 1-aminoanthraquinone, 1-hydroxyanthraquinone, 2-hydroxyanthraquinone, 2-aminoanthraquinone, 2-chloroanthraquinone, 1,5-dihydroxyanthraquinone, 2,6-dihydroxyanthraquinone, 1,8-dihydroxyanthraquinone, and 1,4-diaminoanthraquinone.

11. A method of facilitating odor control in a sewerage system, comprising:
determining the presence of at least one odorous compound in the sewerage system; and
providing an odor control composition consisting essentially of at least one alkaline compound and at least one anthraquinone.

12. The method of claim 11, further comprising providing instructions to adjust the relative ratio of an amount of the at least one alkaline compound to an amount of the at least one anthraquinone.
